(19) United States
(12) Patent Application Publication
(10) Pub. No.: US 2019/0092827 A1
(43) Pub. Date: Mar. 28, 2019

(54) Trapped Chemokine Variants as Therapeutic Agents for Inflammation-Related Diseases Including Infections, Diabetes, and Cancer

(71) Applicant: Board of Regents, The University of Texas System, Austin, TX (US)

(72) Inventors: Krishna Rajarathnam, League City, TX (US); Ashok Chopra, League City, TX (US)

(73) Assignee: Board of Regents, The University of Texas System, Austin, TX (US)

(21) Appl. No.: 16/142,101

(22) Filed: Sep. 26, 2018

Related U.S. Application Data
(60) Provisional application No. 62/564,105, filed on Sep. 27, 2017.

(51) Int. Cl. C07K 14/52

(52) U.S. Cl. CPC C07K 14/522

(57) ABSTRACT

The present invention includes composition and methods of using a recombinant dimeric chemokine covalently modified by introducing a disulfide bond across a dimer interface, wherein the recombinant dimeric chemokine is linked by an intermolecular disulfide bond.

### CROSS-REFERENCE TO RELATED APPLICATIONS

This application claims priority to U.S. Provisional Application Ser. No. 62/564,105, filed Sep. 27, 2017, the entire contents of which are incorporated herein by reference.

### STATEMENT OF FEDERALLY FUNDED RESEARCH

This invention was made with government support under 1P01HL107152 and 1R21AI124681 awarded by the National Institutes of Health. The government has certain rights in the invention.

### TECHNICAL FIELD OF THE INVENTION

The present invention relates in general to the field of trapped chemokines.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Sep. 25, 2018, is named UTBM1047_Seq_listing.txt and is 4 KB in size.

### BACKGROUND OF THE INVENTION

Without limiting the scope of the invention, its background is described in connection with chemokines.

Chemokines play a fundamental role in recruiting different leukocytes in response to infection, in mediating diverse homeostatic responses in various tissues including metabolism, and in propagating cancer.

One such chemokine is taught in U.S. Pat. No. 6,413,510, issued to DeMarsh, et al., entitled “Dimeric modified groβ protein”. These inventors are said to teach certain methods of preventing and treating sepsis using chemokines selected from mature or modified KC, Groα, Groβ, Groγ, or multimers thereof, alone or in conjunction with an anti-infective agent, and includes a groβ dimer chemokine.

The prior art of DeMarsh teaches scrambled intermolecular pairs, rather than intramolecular, specifically at locations C9-C35 and C11-051. Essentially, deMarsh uses variants that involve disulfide formation between 1^{st} (C9) and 4^{th} (C51) Cys and 2^{nd} (C11) and 3^{rd} (C35) Cys. It is not clear whether these scrambled variants were homogenous or mixture of proteins and also how reproducibly they can be produced considering these are misfolded proteins. The final intramolecular disulfide bonds are not described, nor is any receptor activity of the modified variants demonstrated.

Thus, a need remains for improved chemokines that are folded properly and have superior physiological and biochemical characteristics.

### SUMMARY OF THE INVENTION

In one embodiment, the present invention includes a recombinant dimeric chemokine covalently modified by introducing a disulfide bond across the dimer interface, wherein the recombinant dimeric chemokine is linked by an intermolecular disulfide bond. In one aspect, the dimeric chemokine is a human chemokine. In another aspect, the dimeric chemokine is a mouse chemokine. In another aspect, the chemokine is selected from CXCL1, CXCL2, CXCL8, MIP2, KC, IL8, MGSA, or GROb. In another aspect, the recombinant chemokine has an amino acid sequence of SEQ ID NO:1. In another aspect, the recombinant chemokine has an amino acid sequence of SEQ ID NO:2. In another aspect, the recombinant chemokine has an amino acid sequence of SEQ ID NO:3. In another aspect, the recombinant chemokine has an amino acid sequence of SEQ ID NO:4. In another aspect, the recombinant chemokine has an amino acid sequence of SEQ ID NO:5.

In another embodiment, the present invention includes a method of treating a bacterial infection comprising: identifying a subject in need of therapy for a bacterial infection; and providing the subject with an effective amount of a modified recombinant dimeric chemokine, wherein the modified recombinant chemokine proteins are linked by an intermolecular disulfide bond at the dimer interface. In one aspect, the dimeric chemokine is a human chemokine. In another aspect, the dimeric chemokine is a mouse chemokine. In another aspect, the chemokine is selected from CXCL1, CXCL2, CXCL8, MIP2, KC, IL8, MGSA, or GROb. In another aspect, the recombinant chemokine has an amino acid sequence of SEQ ID NO:1. In another aspect, the recombinant chemokine has an amino acid sequence of SEQ ID NO:2. In another aspect, the recombinant chemokine has an amino acid sequence of SEQ ID NO:3. In another aspect, the recombinant chemokine has an amino acid sequence of SEQ ID NO:4. In another aspect, the recombinant chemokine has an amino acid sequence of SEQ ID NO:5. In another aspect, the recombinant chemokine is MIP-2 and the bacterial infection is a Salmonella sp. infection.

In another embodiment, the present invention includes a pharmaceutical composition comprising a recombinant dimeric chemokine comprising a covalent link, wherein the recombinant dimeric chemokine is modified to be linked by an intermolecular disulfide bond. In one aspect, the dimeric chemokine is a human chemokine. In another aspect, the dimeric chemokine is a mouse chemokine. In another aspect, the chemokine is selected from CXCL1, CXCL2, CXCL8, MIP2, KC, IL8, MGSA, or GROb. In another aspect, the recombinant chemokine has an amino acid sequence of SEQ ID NO:1. In another aspect, the recombinant chemokine has an amino acid sequence of SEQ ID NO:2. In another aspect, the recombinant chemokine has an amino acid sequence of SEQ ID NO:3. In another aspect, the recombinant chemokine has an amino acid sequence of SEQ ID NO:4. In another aspect, the recombinant chemokine has an amino acid sequence of SEQ ID NO:5.

In another embodiment, the present invention includes a recombinant chemokine modified to form a disulfide across a dimer interface of the chemokine.

In another embodiment, the present invention includes a recombinant chemokine modified to form a disulfide across a dimer interface of the recombinant chemokine. In one aspect, the recombinant chemokine has SEQ ID NO:1. In another aspect, the recombinant chemokine has SEQ ID NO:2. In another aspect, the recombinant chemokine has SEQ ID NO:3. In another aspect, the recombinant chemokine has SEQ ID NO:4. In another aspect, the recombinant chemokine has SEQ ID NO:5. In another aspect, the recombinant chemokine has forms a dimer.

In another embodiment, the present invention includes a nucleic acid that encodes a recombinant chemokine modified to form a disulfide across a dimer interface of the recombinant chemokine. In one aspect, the nucleic acid encodes an amino acid sequence as set forth in SEQ ID NO: 1, 2, 3, 4, or 5.

### BRIEF DESCRIPTION OF THE DRAWINGS

For a more complete understanding of the features and advantages of the present invention, reference is now made to the detailed description of the invention along with the accompanying figures and in which:

FIG. 1A is a schematic showing native disulfides between 1^{st} and 3^{rd} cysteines and 2^{nd} and 4^{th} cysteines, FIG. 1B is a schematic showing the dimer structure with monomers in different colors and helices in red, and FIG. 1C is a schematic showing the newly introduced disulfide (in white) at the dimer interface.

FIGS. 2A and 2B are graphs that show the results from using the present invention in a mouse model system comparing the native protein and the present invention.

FIG. 3 is an amino acid alignment of the various chemokines for use with the present invention and the location of the cysteine modifications. The amino acid sequence are SEQ ID NOS:1-5, respectively.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The present inventors have created engineered “locked” chemokine dimers that demonstrate beneficial anti-bacterial properties. The invention focuses on locked dimers of chemokine CXCL2 produced using exogenous sequence modifications. While the present inventors have previously published about locked dimer CXCL1, nothing is known on its effects on bacteria. Chemokines naturally exist as monomers and dimers, however, using an exogenous disulfide linkage, the present invention is a variant that is “locked” in a dimer form while still maintaining chemokine structure/function.

While the human chemokines CXCL1 (aka: groα) and CXCL2 (aka: groβ) have been made in modified or multimeric forms to treat sepsis (antibiotic resistant bacterial infection), it has been found that following those teachings the dimeric forms of the chemokines were improperly folded, thus imparting on the chemokine random, non-reproducible internal disulfide bonds and folding errors that render it inactive.

Using the recombinant modified locked chemokine dimers of the present invention, the novel dimers were able to impart protection in a bacterial infection model. The data clearly show that mouse MIP-2 locked dimer is protective, whereas the native MIP-2 did not impart protection and survival. Further, the clinical scores of the mice treated with locked dimer were significantly improved.

By means of explanation, and in no way a limitation of the present invention, the leukocytes recruited in response to locked dimer are poised to destroy pathogens in a fashion that is not possible by native form of these chemokine-mediated leukocytes. Levels of leukocytes and their activation status must be finely tuned to have maximal influence on microbial killing and have minimal collateral damage to the host tissue. It was found that the locked dimers maximize the microbial killing and have minimal collateral damage to the host tissue, and the position of the intermolecular disulfide bonds can be manipulated to enhance or customize clinical relevance. In addition, one or more of the novel recombinant locked dimeric chemokines can be used either alone or in combination with antibiotic or non-antibiotic drugs to combat antibiotic-resistant microbes.

The prior art of deMarsh teaches intermolecular pairs, rather than intramolecular, specifically at locations C9-C35 and C11-051, which uses variants that involve disulfide formation between 1^{st} and 4^{th} Cys and 2^{nd} and 3^{rd} Cys. It is not clear whether these scrambled variants were homogenous or mixture of proteins and also how reproducibly they can be produced considering these are misfolded proteins. The final intramolecular disulfide bonds are not described, nor is any receptor activity of the modified variants demonstrated. In contrast, the present invention intentionally placed an external disulfide bond that maintains chemokine structure/function and locks the dimer. The chemokine variants described in this patent involves the ‘scrambled’ endogenous disulfides. No exogenous modifications were introduced.

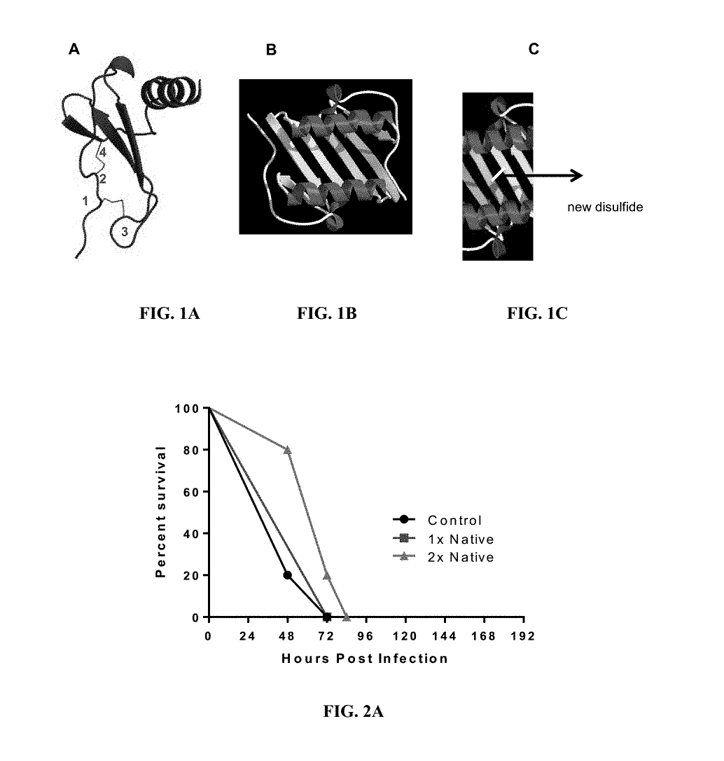

Chemokine CXCL2 (also known as MIP-2 in mice) exists reversibly as monomers and dimers. Structures of the WT MIP-2 dimer are known. Using solution nuclear magnetic resonance (NMR) spectroscopy, the inventors calculated its monomer-dimer equilibrium (K_{d}) as 10 μM. The sequence of MIP-2 reveals four cysteines, a characteristic feature that is conserved in most chemokines. These four cysteines form two disulfides (1^{st} Cys with 3^{rd} Cys and 2^{nd} Cys and 4^{th} Cys) (FIG. 1A). The present inventors and others have previously shown that these disulfides are absolutely critical for structure and function as deleting the disulfides result in loss of structure and significant loss of activity.

In order to unambiguously characterize the activity of the dimer, the present inventors used the strategy of a disulfide-trap to produce a non-dissociating MIP-2 dimer, which involved mutating the residue at the two-fold symmetry to cysteine and expressing the protein by recombinant methods. The newly introduced disulfide bonds did not interfere with the endogenous disulfides and also located at a site away from either of the disulfides. The present inventors confirmed that the design strategy was successful and resulted in a covalently modified trapped dimer from SDS-PAGE electrophoresis carried out under reducing and non-reducing conditions. The present inventors also characterized the solution structure of the trapped MIP-2 dimer, and observed that its structure is essentially identical to that of the native dimer but for subtle local changes due to introduction of a new disulfide bond. The present inventors observed that the trapped MIP-2 dimer is highly efficient in recruiting neutrophils in mouse models.

FIG. 1A is a schematic showing native disulfides between 1^{st} and 3^{rd} cysteines and 2^{nd} and 4^{th} cysteines, FIG. 1B is a schematic showing the dimer structure with monomers in different colors and helices in red, and FIG. 1C is a schematic showing the newly introduced disulfide (in white) at the dimer interface.

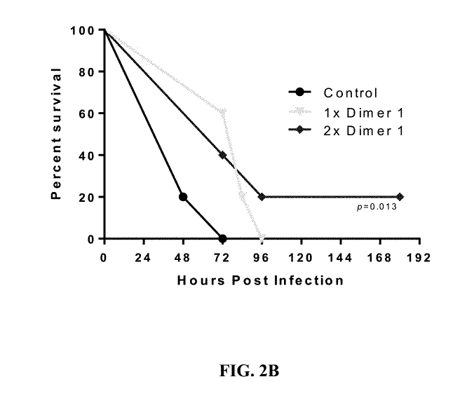

The present inventors characterized the ability of the trapped MIP-2 dimer to impart protection in a Salmonella infection model. FIGS. 2A and 2B compare the wild-type (WT) chemokine (that can exist as both monomers and dimers), with the trapped dimer of the present invention. It was found that, unlike the WT, the trapped dimer of the present invention imparted significant protection to animals infected with a lethal dose of Salmonella. Mortality was significantly reduced and also delayed, and also MIP-2 dimer-treated mice showed significantly lower bacterial counts indicating that neutrophils recruited in response to MIP-2 dimer were more effective in controlling bacterial proliferation due to their killing activity in a timely manner.

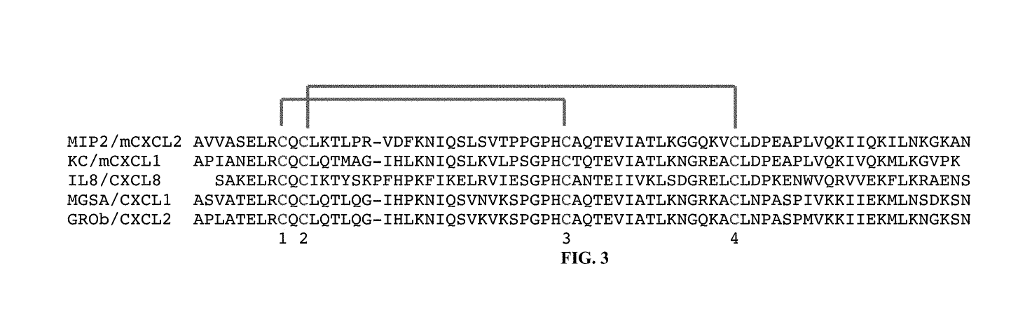

FIG. 3 is an amino acid alignment of the various chemokines for use with the present invention and the location of the cysteine modifications. The amino acid sequence are SEQ ID NOS:1-5, respectively.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A recombinant dimeric chemokine covalently modified by introducing a disulfide bond across a dimer interface, wherein the recombinant dimeric chemokine is linked by an intermolecular disulfide bond.

2. The recombinant dimeric chemokine of claim 1, wherein the dimeric chemokine is a human or a mouse chemokine.

3. The recombinant dimeric chemokine of claim 1, wherein the chemokine is selected from at least one of: CXCL1, CXCL2, CXCL8, MIP2, KC, IL8, MGSA, or GROb.

4. The recombinant dimeric chemokine of claim 1, wherein the recombinant dimeric chemokine has an amino acid sequence of SEQ ID NO:1, 2, 3, 4, or 5.

5. The recombinant dimeric chemokine of claim 1, wherein the recombinant dimeric chemokine is a locked chemokine.

6. A method of treating a bacterial infection comprising:
identifying a subject in need of therapy for a bacterial infection; and
providing the subject with an effective amount of a modified recombinant dimeric chemokine, wherein the modified recombinant chemokine is linked by an intermolecular disulfide bond at the dimer interface.

7. The method of claim 6, wherein the dimeric chemokine is a human or a mouse chemokine.

8. The method of claim 6, wherein the chemokine is MIP-2 dimer and the bacterial infection is a Salmonella sp. infection.

9. The method of claim 6, wherein the chemokine is selected from at least one of: CXCL1, CXCL2, CXCL8, MIP2, KC, IL8, MGSA, or GROβ.

10. The method of claim 6, wherein the recombinant dimeric chemokine has an amino acid sequence of SEQ ID NO:1, 2, 3, 4, or 5.

11. A pharmaceutical composition comprising a recombinant dimeric chemokine comprising a covalent link, wherein the recombinant dimeric chemokine is modified to be linked by an intermolecular disulfide bond.

12. The composition of claim 11, wherein the dimeric chemokine is a human or a mouse chemokine.

13. The composition of claim 11, wherein the chemokine is selected from at least one of: CXCL1, CXCL2, CXCL8, MIP2, KC, IL8, MGSA, or GROβ.

14. The composition of claim 11, wherein the recombinant chemokine has an amino acid sequence of SEQ ID NO:1, 2, 3, 4, or 5.

15. A recombinant chemokine modified to form a disulfide across a dimer interface of the recombinant chemokine.

16. The recombinant chemokine of claim 15, wherein the recombinant chemokine has an amino acid sequence of SEQ ID NO:1, 2, 3, 4, or 5.

17. The recombinant chemokine of claim 15, wherein the recombinant chemokine forms a dimer.

18. A nucleic acid that encodes a recombinant chemokine modified to form a disulfide across a dimer interface of the recombinant chemokine.

19. The nucleic acids of claim 18, wherein the nucleic acid encodes the amino acid sequences as set forth in SEQ ID NO:1, 2, 3, 4, or 5.


SEQUENCE LISTING (5 sequences)

SEQ ID NO: 1 - protein, 73 aa, Artificial Sequence
  Synthetic peptide
AVVASELRCQCLKTLPRVDFKNIQSLSVTPPGPHCAQTEVIATLKGGQKVCLDPEAPLVQKIIQKILNKGKAN

SEQ ID NO: 2 - protein, 72 aa, Artificial Sequence
  Synthetic peptide
APIANELRCQCLQTMAGIHLKNIQSLKVLPSGPHCTQTEVIATLKNGREACLDPEAPLVQKIVQKMLKGVPK

SEQ ID NO: 3 - protein, 72 aa, Artificial Sequence
  Synthetic peptide
SAKELRCQCIKTYSKPFHPKFIKELRVIESGPHCANTEIIVKLSDGRELCLDPKENWVQRVVEKFLKRAENS

SEQ ID NO: 4 - protein, 73 aa, Artificial Sequence
  Synthetic peptide
ASVATELRCQCLQTLQGIHPKNIQSVNVKSPGPHCAQTEVIATLKNGRKACLNPASPIVKKIIEKMLNSDKSN

SEQ ID NO: 5 - protein, 73 aa, Artificial Sequence
  Synthetic peptide
APLATELRCQCLQTLQGIHLKNIQSVKVKSPGPHCAQTEVIATLKNGQKACLNPASPMVKKIIEKMLKNGKSN